(19) United States
(12) Patent Application Publication
(10) Pub. No.: US 2006/0216804 A1
(43) Pub. Date: Sep. 28, 2006

(54) Construction of new xylose utilizing saccharomyces cerevisiae strain

(76) Inventor: Kaisa Johanna Karhumaa, Lund (SE)

Correspondence Address: Gauthier & Connors LLP;Suite 2300, 225 Franklin Street, Boston, MA, 02110, US

(21) Appl. No.: 11/372,644

(22) Filed: Mar. 10, 2006

Related U.S. Application Data
(63) Continuation of application No. PCT/SE04/01237, filed on Aug. 30, 2004.

(30) Foreign Application Priority Data
Sep. 11, 2003 (SE) 0302421-3

(51) Int. Cl. C12P 7/06; C12N 1/18

(52) U.S. Cl. 435/161; 435/254.21

(57) ABSTRACT

The present invention relates to a novel Saccharomyces cerevisiae strain utilizing xylose for fermenting ethanol expressing xylose isomerase (XI), overexpressing xylulokinase (XK), overexpressing the pentose phosphate pathway (PPP), and non-expressing aldose reductase (AR) and being adapted to growth in mineral defined medium with xylose as sole carbon source.

The present invention relates to a novel Saccharomyces cerevisiae strain producing ethanol from xylose containing medium.

### BACKGROUND OF THE INVENTION

Production of ethanol for use as e.g., fuel or fuel additive from carbohydrate feedstocks, such as hydrolysates of plants can be made by ethanolic fermentation using yeasts, such as Saccharomyces cerevisiae. As such feedstocks may comprise pentoses, such as xylose there is a demand for Saccharomyces cerevisiae strains that can convert not only hexoses but also pentoses such xylose.

Lignocellulose is the main component of forest product residues and agricultural waste. Lignocellulosic raw materials are mainly composed of cellulose, hemicellulose, and lignin. The cellulose fraction is made up of glucose polymers, whereas the hemicellulose fraction is made up of a mixture of glucose, galactose, mannose, xylose, and arabinose polymers. The lignin fraction is a polymer of phenolic compounds.

The cellulose and hemicellulose fractions can be hydrolyzed to monomeric sugars, which can be fermented to ethanol. Ethanol can serve as an environmentally friendly liquid fuel for transportation, since carbon dioxide released in the fermentation and combustion processes will be taken up by growing plants in forests and fields.

The price for lignocellulose-derived ethanol has been estimated by von Sivers et al. (“Cost analysis of ethanol production from willow using recombinant Escherichia coli, Biotechnol. Prog. 10:555-560, 1994). The calculations are based on the fermentation of all hexose sugars (glucose, galactose, and mannose) to ethanol. It was estimated that the fermentation of pentose sugars (xylose and arabinose) to ethanol will reduce the price of ethanol by approximately 25%.

Xylose is found in hardwood hemicellulose, whereas arabinose is a component in hemicellulose in certain agricultural crops, such as corn. In order to make the price of ethanol more competitive, the price must be reduced.

The release of monomeric sugars from lignocellulosic raw materials also releases by-products, such as weak acids, furans, and phenolic compounds, which are inhibitory to the fermentation process. Numerous studies have shown that the commonly used Baker's yeast, Saccharomyces cerevisiae, is the only ethanol producing microorganism that is capable of efficiently fermenting non-detoxified lignocellulose hydrolysates (Olsson and Hahn-Hägerdal, “Fermentation of lignocellulosic hydrolysates for ethanol production”, Enzyme Mjcrobial Technol. 18:312-331 (1996). Particularly efficient fermenting strains of S. cerevisiae been isolated from the fermentation plant at a pulp and paper mill (Linden et al., “Isolation and characterization of acetic acid-tolerant galactose-fermenting strains of Saccharomyces cerevisiae from a spent sulfite liquor fermentation plant”, Appl. Envjron. Mjcrobjol. 58:1661-1669, 1992).

S. cerevisiae ferments the hexose sugars glucose, galactose and mannose, but is unable to ferment the pentose sugars xylose and arabinose due to the lack of one or more enzymatic steps. S. cerevisiae can ferment xylulose, an isomerisation product of xylose, to ethanol (Wang et al., “Fermentation of a pentose by yeasts”, Biochem. Biophys. Res. Commun. 94:248-254, 1980; Chiang et aJ., “D-Xylulose fermentation to ethanol by Saccharomyces cerevisiae”, Appl. Environ. Microbiol. 42:284-289, 1981; Senac and Hahn-Hägerdal, “Intermediary metabolite concentrations in xylulose- and glucose-fermenting Saccharomyces cerevisiae cells”, Appl. Environ. Microbiol. 56:120-126, 1990).

In eukaryotic cells, the initial metabolism of xylose is catalyzed by a xylose reductase (XR), which reduces xylose to xylitol, and a xylitol dehydrogenase (XDH), which oxidizes xylitol to xylulose. Xylulose is phosphorylated to xylulose 5-phosphate by a xylulose kinase (XK) and further metabolized through the pentose phosphate pathway and glycolysis to ethanol. S. cerevisiae has been genetically engineered to metabolize and ferment xylose via this pathway. The genes for XR and XDH from the xylose fermenting yeast Pichia stipitis have been expressed in S. cerevisiae (European Patent to C. Hollenberg. 1991; Hallborn et al., “Recombinant yeasts containing the DNA sequences coding for xylose reductase and xylitol dehydrogenase enzymes”, WO91/15588; Kbtter and Ciriacy, “Xylose fermentation by Saccharomyces cerevisiae”, Appl. Microbiol. Biotechnol. 38:776-783, 1993). The transformants metabolize xylose but do not ferment the pentose sugar to ethanol.

When the gene for the enzyme transaldolase (TAL) is overexpressed in xylose-metabolizing transformants, the new recombinant strains grow better on xylose but still do not produce any ethanol from xylose (Walfridsson et al., “Xylose-metabolizing Saccharomyces cerevisiae strains overexpressing the TKL1 and TAL1 genes encoding the pentose phosphate pathway enzymes transketolase and transaldolase”, Appl. Environ. Microbiol. 61:4184-4190, 1995). In these strains, the major metabolic by-product, in addition to cell mass, is xylitol formed from xylose through the action of the enzyme XR. When the expression of XDH is ten times higher than the expression of XR, xylitol formation is reduced to zero (Walfridsson et al., “Expression of different levels of enzymes from Pichia stipitis XYL1 and XYL2 genes in s and its effect on product formation during xylose utilization”. Appl. Microbiol. Biotechnol. 48:218-224, 1997). However, xylose is still poorly fermented to ethanol.

The gene for xylulose kinase (XK) from S. cerevisiae has been cloned and overexpressed in XR-XDH-expressing transformants of S. cerevisiae (Deng and Ho, ‘Xylulokinase activity in various yeasts including Saccharomyces cerevisiae containing the cloned xylulokinase gene”, Appl. Biochem. Biotechnol. 24125.193-199, 1990. Ho and Tsao, “Recombinant yeasts for effective fermentation of glucose, and xylose”, WO95/13362, 1995; Moniruzzaman et al., “Fermentation of corn fibre sugars by an engineered xylose utilizing Saccharomyces strain”, World J. Microbiol. Biotechnol. 13:341-346, 1997). These strains have been shown to produce net quantities of ethanol in fermentations of mixtures of xylose and glucose. Using the well established ribosomal integration protocol, the gene have been chromosomally integrated to generate strains that can be used in complex media without selection pressure (Ho and Chen, “Stable recombinant yeasts for fermenting xylose to ethanol”, WO97/42307. Toon et al., “Enhanced cofermentation of glucose and xylose by recombinant Saccharomyces yeast strains in batch and continuous operating modes”, Appl. Biochem. Biotechnol. 63/65:243-255, 1997).

In prokaryotic cells, xylose is isomerized to xylulose by a xylose isomerase (XI). Xylulose is further metabolized in the same manner as in the eukaryotic cells. XI from the thermophilic bacterium Thermus thermophilus was expressed in S. cerevisiae, and the recombinant strain fermented xylose to ethanol (Walfridsson et aJ., “Ethanolic fermentation of xylose with Saccharomyces cerevisiae harboring the Thermus thermophilus xylA gene which expresses an active xylose (glucose) isomerase”, Appl. Environ. Microbiol. 62:4648-4651, 1996). The low level of ethanol produced was assumed to be due to the fact that the temperature optimum of the enzyme is 85° C., whereas the optimum temperature for yeast fermentation is 30° C.

Saccharomyces cerevisiae as such can thus not ferment xylose, but has to be modified. Thus one way is to overexpress the genes coding for xylose reductase (XR), xylitol dehydrogenase (XDH) and xylulokinase (XK), whereby and isomerisation product of xylose, viz. xylulose, is obtained.

Another way is to overexpress xylose isomerase (XI), whereby xylose is directly converted to xylulose. (Träff et al, Appl Environ Microbiol 2001:67(12):5668-74; Lönn et al, Enz Microbiol Tech, 2003:32:567-573). Hereby a recombinant S. cerevisiae strain comprising mutated xylA from Thermus thermophilus is construed.

Kuyper et al, FEMS Yeast Res 2003:1574:1-10 discloses high-level functional expression of fungal xylose isomerase derived from Piromyces xylose isomerase gene. The strain construed was not shown to grow anaerobically or aerobically on a glucose-xylose medium but show a small xylose uptake. The strain grew on sole xylose with a growth rate of 0.005.

However, this strain utilizes a combined glucose-xylose medium, and seems not to be adapted to a mere xylose medium.

There is thus a demand for a xylose fermenting strain expressing xylose isomerase and having an improved growth rate and improved ethanol yield.

### SUMMARY OF THE INVENTION

In accordance with the present invention a new Saccharomyces cerevisiae strain has been construed solving this problem. The strain comprises a xylose isomerase (XI) expressing gene xylA disclosed in Lönn et al (supra) but also present in plasmid pBXI, an overexpression of xylulokinase (XK), an overexpression of the pentose phosphate pathway, having a deleted GRE3 gene (Träff et al, supra) and being adapted to growth in mineral defined medium with xylose as the sole carbon source.

The XI used originated from a plasmid PBXI, and is thus a wild-type XI.

This Saccharomyces cerevisiae strain denoted TMB 3050, has been deposited at Deutsche Sammlung von Mikroorganismen und Zellkulturen on the 14^{th} of August 2003, under deposition number DSM 15834.

### DETAILED DESCRIPTION OF THE INVENTION

The present invention thus claims a Saccharomyces cerevisiae strain expressing xylose isomerase (XI), overexpressing xylulokinase (XK), overexpressing the pentose phosphate pathway, non-expressing aldose reductase (AR) and being adapted to growth in mineral defined medium with xylose as sole carbon source.

In particular the strain expresses xylose isomerase derived from xylA gene. Overexpression of xylulokinase is obtained by adding a plasmid expressing XKS1 (Lönn et al. 2003) coding for xylulokinase. Overexpression of the pentose phosphate pathway is obtained by adding extra copies of the genes TAL1, TKL1, RPE1, RKI1 (Johansson & Hahn-Hägerdal 2002). Non-expression of aldose reductase (AR) is obtained be deleting the gene GRE3, to reduce formation of xylitol.

The construction of the strain will be described more in detail in the following.

Methods

Standard molecular biology techniques were used. (Sambrook et al. 1989) Lithium acetate method was used for yeast transformation (Gietz & Schiestl 1995). Yeast chromosomal DNA was extracted with Easy-DNA Kit (Invitrogen). Colony PCR was performed using the Lyse-N-Go reagent (Pierce, Rockford, Ill., USA). PCR was performed with the following program: 95° C. for 5 min, 45 cycles of 95° C. for 30 s, 45° C. for 30 s and 72° C. for 1 min 20 s, 72° C. for 7 min. In colony PCR and PCR on chromosomal yeast DNA, Taq-polymerase (Fermentas) was used. In other PCR reactions, PWO polymerase (Roche) was used.

Media and Cultivation Conditions

Yeast was grown on YPD medium (20 g/l peptone, 10 g/l yeast extract and 20 g/l glucose), SC medium (6,7 g/l Difco Yeast Nitrogen Base, 20 g/l glucose or 20 g/l galactose) or defined mineral medium (Verduyn et al. 1990). The amount or sugar used in mineral medium was 20 g/l glucose or 50 g/l xylose. 2.042 g phthalate and 0.301 g NaOH was added to mineral medium, and pH was set to 5.5 before sterilization. Amino acids were added to defined mineral medium when necessary. The amino acid concentrations used were: 20 μg/ml histidine, 20 μg/ml tryptophan, 240 μg/ml leucine and 20 μg/ml uracil. The cultures were grown in baffled shake flasks with 130 rpm shaking.

Plate cultures were grown on YPD-agar plates (20 g/l peptone, 10 g/l yeast extract and 20 g/l glucose, 20 g/l agar) or YNB-plates (6,7 g/l Difco Yeast Nitrogen Base, 30 g/l agar and 20 g/l glucose or 50 g/l xylose). Zeocin (Invitrogen, Groningen, The Netherlands) was added to YPD plates at 50 mg/l.

Construction of TMB 3044

The Saccharomyces cerevisiae strain YUSM1009a (Träff et al. 2001) was transformed with plasmid YIpXK (Lönn et al. 2003) linearized with NdeI. Transformants were selected on YNB-plates containing uracil, leucin and tryptophan but not histidine. Chromosomal integration of the plasmid was confirmed by colony PCR and PCR on chromosomal DNA with primers BJ0697 and BJ5756. The overexpression of the gene coding for xylulokinase (XK) was confirmed with enzyme assay.

The resulting strain was transformed with plasmid pB3PGK TAL1 (johansson & Hahn-Hägerdal 2002) linearized with BglII. Transformants were selected on YPD plates containing zeocin. Chromosomal integration of the plasmid was confirmed by colony PCR and PCR on chromosomal DNA with primers BJ5756 and 3TAL1clon.

To remove the zeocin marker, the resulting strain was transformed with plasmid pCRE3 (Johansson & Hahn-Hägerdal 2002). The transformants were selected on YNB plates containing leucin and tryptophan, but not uracil. The resulting transformant was grown in 500 ml shake flask in 100 ml SC-medium containing galactose for about 24 h. To remove the plasmid, 1 ml aliquot of the culture was inoculated to 100 ml YPD medium in 500 ml shake flask and grown for 24 h. An aliquot of the culture was plated on a YPD plate. Zeocin-sensitive colonies were selected by replica plating on a YPD plate containing zeocin. One zeocin sensitive clone was purified by repeated plating on YPD plates.

The resulting clone was transformed with pB3PGK RKI1 (Johansson & Hahn-Hägerdal 2002) linearized with BcuI (Fermentas). Chromosomal integration of the plasmid was confirmed by colony PCR with primers BJ5756 and 3RKI1clon. The zeocin marker was removed same way as before.

The resulting clone was transformed with pB3PGK TKL1 (Johansson & Hahn-Hägerdal 2002) linearized with BshTI (Fermentas). Chromosomal integration of the plasmid was confirmed by colony PCR with primers BJ5756 and 3TKL1clon. The zeocin marker was removed same way as before. Overexpression of the pentose phosphate pathway is thereby obtained.

The resulting clone was transformed with pB3PGK RPE1 (Johansson & Hahn-Hägerdal 2002) partially digested with XcmI (New England Biolabs). Chromosomal integration of the plasmid was confirmed by colony PCR with primers BJ5756 and 3RPE1clon. The zeocin marker was removed same way as before.

Tryptophan auxotrophy in the resulting strain was cured by transforming with product from a PCR with primers TRP5 and TRP3 and the plasmid YEplac112 as a template. The transformants were selected on a YNB plate lacking tryptophan.

In the resulting strain, leucin auxotrophy was cured by transforming with the plasmid YEplac181 linearized with ScaI. The transformants were selected on a YNB plate lacking leucin. The resulting strain was named TMB 3044.

Plasmid Construction

A cassette of HXT7 truncated promoter and PGK terminator was digested from plasmid pHM96 (Hauf et al. 2000) with Sad and HindIII. The resulting fragment was cloned in YEplac195 linearized with SacI and HindIII. The resulting plasmid was named YEplacHXT.

The xylose isomerase gene xylA of Thermus thermophilus was amplified by PCR using primers prBCL and terPST and plasmid pBXI (Walfridsson et al. 1996) as a template. The product was digested with BclI and PstI and cloned in plasmid YEplacHXT linearized with BamHI and PstI. The resulting plasmid was named YEplacHXT-XI to express xylose isomerase when inserted.

The resulting plasmid was named YEplacHXT-XI, having T. thermophilus xylA gene downstream of the HXT7-truncated promoter for highest possible expression of T. thermophilus XI.

Construction of TMB3050

Plasmid YEplacHXT-XI was transformed to TMB 3044. Transformants were selected on YNB plates lacking uracil. One of the transformants was purified by repeated plating on YNB plates. The purified transformant was grown in mineral medium containing glucose and an aliquot of the culture was plated on an YNB plate containing 50 g/l xylose as a sole carbon source. After two months of incubation at 30° C., about 20 of the ˜1000 colonies on the plate appeared larger than others. One of these colonies was purified by repeated plating on a YNB plate containing 50 g/l xylose. After purification, the strain was grown in mineral medium (50 g/l xylose, no phthalate, no NaOH) for 4 weeks. An aliquot of the culture was reinoculated to fresh medium and the culture was incubated for two weeks. When an aliquot of this culture was re-inoculated, the culture reached in three days stationary phase at optical density (620 nm) of 7.7. An aliquot of this culture was purified by repeated plating.

This culture was named TMB 3050. When grown on mineral medium containing 50 g/l xylose, buffered with phthalate and NaOH, the strain grows with maximal growth rate of 0.12-0.14 and reaches optical density of >15 in about 3 days (FIG. 1).

FIG. 2 shows the gene construct of the present strain

The present strain was compared with the strains according to Kuyper et al (literature comparison) and the strain of Lönn et al (supra) as to aerobic growth, and anaerobic growth.

TABLE 1

| | | TMB3050 | Lönn et al | Kuyper et al |
|---|---|---|---|---|
| AEROBIC | Growth rate | 0.12-0.14 | n.a. | 0.005 |
| | Xylose uptake rate | 0.1466 | n.a. | 0.0495 |
| | (g xylose/g cells/h) | | | |
| | XI activity | 0.23 | 0.012 | 0.4-1.1 |
| | (U/mg cell extract) | (50° C.) | (30° C.) | (30° C.) |
| ANAEROBIC | Temperature at | 30° C. | 40° C. | 30° C. |
| | fermentation | | | |
| | Type of fermentation | High cell density | High cell density | Chemostat |
| | Sugar composition | 40 g/l xylose | 50 g/l xylose | 20 g/l glucose + |
| | in media | | | 10 g/l xylose |
| | Xylose uptake rate | 0.0034 | 0.0043 | 0.109 |
| | (g xylose/g cells/h) | | | |
| | Ethanol yield/ | 0.0439 | n.a. | n.a. |
| | total xylose | | | |
| | Ethanol yield/ | 0.389 | 0.43 | n.a. |
| | consumed xylose | (wt XI) | (mutant 1021) | |
| | Xylitol yield/ | 0.365 | 0 | n.a. |
| | consumed xylose | | | |

n.a. = not available information

As evident from Table 1 above the growth rate of the present strain is 26 times higher than the growth rate of Kuyper et al, and produces ethanol at 30° C., which the Kuyper et al strain does not.

TABLE 2
Specific xylose uptake rates (q_{xylose}), ethanol and xylitol yield
coefficients (Yₑₜₕₐₙₒₗ and Y_{xylitol}) and specific ethanol
productivities (qₑₜₕₐₙₒₗ) from aerobic and anaerobic batch
cultivations of S. cerevisiae strains TMB 3050 and TMB 3045
(=TMB 3044 + XI).

| | Aerobic | Anaerobic | | | |
|---|---|---|---|---|---|
| Strain | q_{xylose}^{b} | q_{xylose}^{b} | Yₑₜₕₐₙₒₗ^{c} | Y_{xylitol}^{d} | qₑₜₕₐₙₒₗ^{e} |
| TMB 3050 | 0.16 ± 0.017 | 0.0049 ± 0.0013 | 0.029 ± 0.013 | 0.031 ± 0.009 | 0.0012 ± 0.0001 |
| TMB 3044 + XI | 0 | 0 | 0 | 0 | 0 |

^{a}h⁻¹
^{b}g xylose g cells⁻¹ h⁻¹
^{c}g ethanol g xylose consumed⁻¹
^{d}g xylitol g xylose consumed⁻¹
^{e}g ethanol g cells⁻¹ h⁻¹

TABLE 3
Specific XI activities in cell extracts, measured at 50° C.

| Strain | 50° C. |
|---|---|
| TMB 3044 + XI (YEplacHXT-XI) | 0.188 ± 0.017 |
| TMB 3044 + plasmid YEplac195 | 0.004 ± 0.003 |
| TMB 3050 (grown in glucose) | 0.095 ± 0.017 |
| TMB 3050 (grown in xylose) | 0.153 ± 0.031 |

### FIGURE LEGENDS

FIG. 1. Aerobic growth of mutant strain TMB 3050( ) and parental strain TMB 3044 with XI (Δ) in defined mineral medium with 50 g/l xylose as the sole carbon source. TMB 3044 with XI was pre-cultured in defined mineral medium containing glucose and TMB 3050 was pre-cultured in defined mineral medium containing xylose.

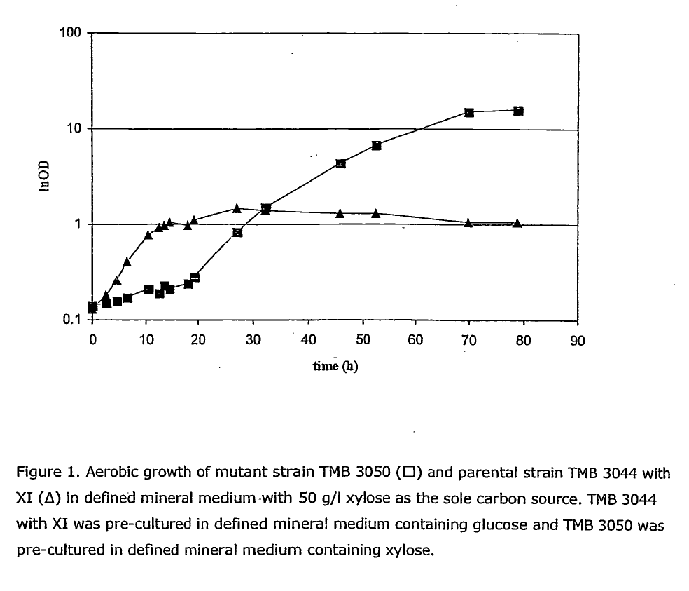

FIG. 2. The gene construct of the present strain

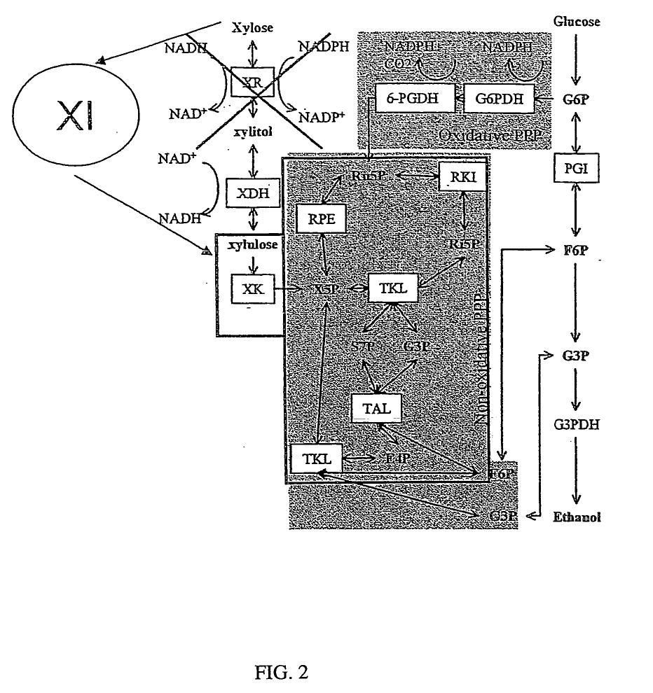

1. A saccharomyces cerevisiae strain utilizing xylose for fermenting ethanol expressing xylose isomerase (XI), overexpressing xylulokinase (XK), overexpressing the pentose phosphate pathway (PPP), and non-expressing aldose reductase (AR) and being adapted to growth in mineral defined medium with xylose as sole carbon source.

2. A saccharomyces cerevisiae strain according to claim 1, wherein the strain expresses xylose isomerase derived from a Thermus thermophilus xylA gene, overexpresses xylulokinase by an addition of a plasmid YIpXK (Lönn et al. 2003) linearized with NdeI coding for xylulokinase, overexpresses the pentose phosphate pathway by adding the genes TAL1, TKL1, RPE1, RKI1, and non-expresses aldose reductase by deletion of the gene GRE3 and being adapted to growth in mineral defined medium with xylose as sole carbon source.

3. A saccharomyces cerevisiae strain according to claim 1, wherein the strain exhibits a growth rate of at least 0.12 h⁻¹, and a xylose uptake rate of at least 0.10 g xylose/g cells/h.

4. A Saccharomyces cerevisiae strain according to claim 1, wherein the strain is TMB3050 deposited at Deutsche Sammlung von Mikroorganismen und Zellkulturen under deposition number DSM 15834.
